(19) United States
(12) Patent Application Publication
(10) Pub. No.: US 2019/0387979 A1
(43) Pub. Date: Dec. 26, 2019

(54) BLOOD PRESSURE MEASUREMENT APPARATUS, METHOD, AND PROGRAM

(71) Applicant: OMRON HEALTHCARE CO., LTD., Muko-shi (JP)

(72) Inventors: Ayako KOKUBO, Kyoto (JP); Hirotaka WADA, Kyoto (JP); Yousuke INAI, Kyoto (JP)

(73) Assignee: OMRON HEALTHCARE CO., LTD., Muko-shi (JP)

(21) Appl. No.: 16/561,728

(22) Filed: Sep. 5, 2019

Related U.S. Application Data
(63) Continuation of application No. PCT/JP2018/009579, filed on Mar. 12, 2018.

(30) Foreign Application Priority Data
Mar. 15, 2017 (JP) 2017-050650

(51) Int. Cl. A61B 5/022; A61B 5/021

(52) U.S. Cl. CPC A61B 5/022; A61B 5/02007; A61B 5/02108; A61B 5/02141

(57) ABSTRACT

The present invention provides a blood pressure measurement apparatus that can judge a feature of a biological body based on biological information. The blood pressure measurement apparatus includes a blood pressure measurement unit that obtains time-series data indicating a blood pressure value that changes in conjunction with a heartbeat, a detection unit that detects one or more surge sections that each include one surge from the time-series data, an extraction unit that extracts one or more feature amounts for each surge, and a classification unit that classifies each of the surges based on the one or more feature amounts.

### TECHNICAL FIELD

This invention relates to a blood pressure measurement apparatus, method, and program for performing continuous blood pressure value measurement.

### BACKGROUND ART

Developments in sensor technology have created an environment in which high-performance sensors can be used easily, and therefore the medical field has seen a gradual increase in the importance of being able to utilize biological information to make early detections of abnormalities in biological bodies and perform treatment.

There is known to be a tonometry type of blood pressure measurement apparatus that can measure biological information such as a pulse or a blood pressure with use of information detected by a pressure sensor that has been placed in direct contact with a biological body at a site having an artery such as a radial artery in a wrist (e.g., see JP 2017-006672A).

### SUMMARY OF INVENTION

However, the blood pressure measurement apparatus disclosed in JP 2017-006672A can merely acquire blood pressure information from the biological body, and cannot detect an abnormality in such blood pressure information.

This invention was achieved in light of the aforementioned circumstances, and an object of the invention is to provide a blood pressure measurement apparatus, method, and program that can extract a feature amount regarding a blood pressure surge, which is a sudden change in blood pressure (hereinafter, called a “surge”), from blood pressure information.

A first aspect of this invention for solving the above-described problem is a blood pressure measurement apparatus including: a blood pressure measurement unit configured to obtain time-series data indicating a blood pressure value that changes in conjunction with a heartbeat; a detection unit configured to detect one or more surge sections that each include one surge from the time-series data; an extraction unit configured to extract one or more feature amounts for each of the surges; and a classification unit configured to classify each of the surges based on the one or more feature amounts.

In a second aspect of this invention, the classification unit maps each of the surges in a space, the number of dimensions of the space being greater than or equal to 1 and less than or equal to the number of feature amounts, and each axis of the space corresponding to one of the feature amounts.

In a third aspect of this invention, the extraction unit extracts, as the one or more feature amounts for each of the surges, a rise time from when the surge starts until when the surges reaches a peak, a fall time from when the surge reaches the peak until when the surge ends, and an amount of change of the blood pressure in the rise time.

In a fourth aspect of this invention, letting a peak point be a local maximum value of at least one of a systolic blood pressure, a diastolic blood pressure, an average blood pressure, and a pulse pressure in the time-series data, letting a start point be a closest local minimum value before the peak point, and letting an end point be a point that is before a closest local minimum value after the peak point and that is when a difference from the closest local minimum value after the peak point has fallen to a certain value or lower, in a case where a difference between a blood pressure value at the peak point and a blood pressure value at the start point is greater than a threshold value, furthermore a time difference between the peak point and a start point prior thereto is greater than a first duration, and still furthermore a time difference between the peak point and the end point is greater than or equal to a second duration, the detection unit detects that a section from the start point to the end point with the peak point therebetween is a surge section.

In a fifth aspect of this invention, the apparatus further includes: a visualization unit configured to visualize each of the surges classified by the classification unit with use of the one or more feature amounts as one or more axes.

In a sixth aspect of this invention, the apparatus further includes: a calculation unit configured to calculate a statistic regarding the one or more feature amounts.

In a seventh aspect of this invention, the apparatus further includes: a visualization unit configured to visualize each of the surges classified by the classification unit with use of the statistic regarding the one or more feature amounts as one or more axes.

In an eighth aspect of this invention, the apparatus further includes: a judgment unit configured to judge a level of danger of a biological body based on a threshold value for comparison with the statistic, the threshold value being associated with a level of danger that is set in advance in correspondence with the statistic.

In a ninth aspect of this invention, the apparatus further includes: a visualization unit configured to visualize each of the surges in accordance with the level of danger, wherein a danger reference corresponding to the threshold value is set in order to judge whether or not a surge is dangerous.

In a tenth aspect of this invention, the apparatus further includes: a judgment unit configured to judge whether a detected surge corresponds to a time in a dosage period based on a dosage record.

In an eleventh aspect of this invention, the apparatus further includes: a visualization unit configured to visualize each of the surges in accordance with whether or not the surge corresponds to a time in the dosage period.

In a twelfth aspect of this invention, the blood pressure measurement unit obtains the time-series data for each of a plurality of users, and the visualization unit visualizes each of the surge for each of the users.

In a thirteenth aspect of this invention, the apparatus further includes: a frequency calculation unit configured to calculate an occurrence frequency of the surges; and an index calculation unit configured to calculate an apnea-hypopnea index based on the occurrence frequency with reference to a relationship between the surge occurrence frequency and an apnea-hypopnea index, the relationship being acquired in advance.

According to the first aspect of this invention, the blood pressure measurement apparatus obtains time-series data indicating a blood pressure value that changes in conjunction with a heartbeat, detects one or more surge sections that each include one surge from the time-series data, extracts one or more feature amounts for each of the surges, and classifies each of the surges based on the one or more feature amounts, thus obtaining a feature regarding surges of the biological body from which the blood pressure values were acquired. If the biological body is determined to have an illness based on the feature, it is possible to judge whether or not the biological body needs medicine or whether there is a need for more thorough examination.

According to the second aspect of this invention, the classification unit maps each of the surges in a space, the number of dimensions of the space being greater than or equal to 1 and less than or equal to the number of feature amounts, and each axis of the space corresponding to one of the feature amounts. Accordingly, the surge features are mapped in a space, and therefore it is easy to classify features according to the regions in the space, and surge features can be understood immediately. Accordingly, features of the biological body that are causing surges can also be understood.

According to the third aspect of this invention, the extraction unit extracts, as the one or more feature amounts for each of the surges, a rise time from when the surge starts until when the surges reaches a peak, a fall time from when the surge reaches the peak until the surge ends, and an amount of change of the blood pressure in the rise time, and therefore the surges can be mapped in a two-dimensional space that has the rise time and the fall time as axes. Accordingly, the surges can be classified according to the rise time and the fall time, and a feature of the biological body can be understood based on these two indicators of the biological body. Furthermore, the extent of the burden on the respiratory system can also be estimated based on the change amount, and the surges can be mapped in a three-dimensional space that also includes the change amount as an axis. As a result, a feature of the biological body can be understood based on these three indicators of the biological body.

According to the fourth aspect of this invention, letting a peak point be a local maximum value of the systolic blood pressure in the time-series data, letting a start point be a closest local minimum value before the peak point, and letting an end point be a point that is before a closest local minimum value after the peak point and that is when a difference from the closest local minimum value after the peak point has fallen to a certain value or lower, in a case where the difference between the blood pressure value at the peak point and the blood pressure value at the start point is greater than a threshold value, furthermore the time difference between the peak point and the start point is greater than the first duration, and still furthermore the time difference between the peak point and the end point is greater than or equal to a second duration, the detection unit detects that a section from the start point to the end point with the peak point therebetween is a surge section. Accordingly, it is possible to clearly see which portion of the blood pressure value time-series data corresponds to a surge, and the blood pressure measurement apparatus can accurately classify the surges.

According to the fifth aspect of this invention, the surges classified by the classification unit are visualized using the feature amounts as axes, thus enabling a doctor or a patient to view the classified surges based on the feature amounts. Accordingly, the surge-related state of the patient can be understood at a glance.

According to the sixth aspect of this invention, a statistic regarding the one or more feature amounts is calculated, thus making it easier to understand an overall trend in a certain time period.

According to the seventh aspect of this invention, the statistics are used to visualize the surges, thus making it easier to understand an overall trend in a certain time period at a glance.

According to the eighth aspect of this invention, it is possible to judge the level of danger of the biological body by comparing the statistic with a threshold value that is associated with a level of danger, and therefore if statistics are obtained for multiple patients, it is possible to more easily understand a standard of the level of danger of each patient.

According to the ninth aspect of this invention, the surges are visualized in accordance with the level of danger, and a danger reference is set in order to judge whether or not a surge is dangerous, thus making it possible to find out the level of danger for each surge. Also, in the case where there are multiple users, it is possible to find out the level of danger for each user.

According to the tenth aspect of this invention, it is possible to judge whether or not a detected surge corresponds to a time in a dosage period, thus making it possible to associate surge feature amounts with the presence or absence of a dosage and give consideration to such information. If a change in the state of a patient is detected based on the surge feature amount, it is possible to judge the efficacy of a dosage.

According to the eleventh aspect of this invention, the surges are visualized according to whether or not they correspond to times in a dosage period, thus making it possible to determine whether or not a surge change occurred in a dosage period. It is therefore possible to judge at a glance whether or not a dosage has had an effect on a surge.

According to the twelfth aspect of this invention, surges are visualized for each user, thus making it possible to understand surge features for each user.

According to the thirteenth aspect of this invention, the occurrence frequency of blood pressure surges is calculated, and an apnea-hypopnea index is calculated based on a pre-acquired relationship between the blood pressure surge occurrence frequency and the apnea-hypopnea index, and therefore the apnea-hypopnea index can be calculated by referencing the blood pressure surges. Accordingly, the apnea-hypopnea index can be calculated by analyzing blood pressure surges, thus making it possible to more easily obtain this index than conventionally possible.

In other words, according to the above aspects of this invention, it is possible to provide a blood pressure measurement apparatus, method, and program that make it possible to judge a feature of a biological body based on biological information.

### BRIEF DESCRIPTION OF DRAWINGS

FIG. 1 is a block diagram showing a blood pressure measurement apparatus according to a first embodiment.

FIG. 2 is a block diagram showing a blood pressure measurement unit included in the blood pressure measurement apparatus in FIG. 1.

FIG. 3 is a diagram showing an example in which the blood pressure measurement apparatus in FIG. 1 has been attached to a wrist.

FIG. 4 is a cross-sectional view of the wrist to which the blood pressure measurement apparatus in FIG. 3 has been attached.

FIG. 5 is a diagram showing an example of an arrangement of sensors in FIGS. 2 to 4.

FIG. 6 is a diagram showing surge feature amounts.

FIG. 7 is a diagram showing time change in the pressure pulse wave pressures of respective heartbeats, and one of the pulse waves.

FIG. 8 is a diagram showing continuous blood pressure value time-series data in which surges are observed.

FIG. 9 is a diagram showing a two-dimensional map regarding a feature amount of the surges in FIG. 8.

FIG. 10 is a flowchart showing operations of the blood pressure measurement apparatus in FIG. 1.

FIG. 11 is a block diagram showing a blood pressure measurement apparatus according to a second embodiment.

FIG. 12 is a diagram showing data stored in a surge detection result DB in FIG. 11.

FIG. 13 is a diagram showing data stored in a surge statistic DB in FIG. 11.

FIG. 14 is a diagram showing data stored in a danger threshold value DB in FIG. 11.

FIG. 15 is a diagram showing data stored in a dosage record DB in FIG. 11.

FIG. 16 is a flowchart showing operations in Example 1 in FIG. 11.

FIG. 17 is a diagram showing an example of a display in a case where a visualization unit has plotted one type of feature amount on one axis in Example 1.

FIG. 18 is a diagram showing an example of a display in a case where the visualization unit has plotted two types of feature amounts on two axes in Example 1.

FIG. 19 is a flowchart showing operations in Example 2 in FIG. 11.

FIG. 20 is a diagram showing an example of a display in a case where the visualization unit has plotted two types of feature amounts on two axes in Example 2.

FIG. 21 is a flowchart showing operations in Example 3 in FIG. 11.

FIG. 22 is a diagram showing an example of a display in a case where the visualization unit has plotted two types of feature amounts on two axes in Example 3.

FIG. 23 is a flowchart showing operations in Example 4 in FIG. 11.

FIG. 24 is a diagram showing an example of a display in a case where the visualization unit has plotted two types of feature amounts on two axes in Example 4.

FIG. 25 is a block diagram showing a blood pressure measurement apparatus according to a third embodiment.

FIG. 26 is a diagram showing a portion of content in an AHI-SI correlation DB in FIG. 25.

FIG. 27 is a flowchart showing operations of the blood pressure measurement apparatus in FIG. 25.

FIG. 28 is a block diagram showing an example of implementation of the blood pressure measurement apparatus according to one of the embodiments.

### DESCRIPTION OF EMBODIMENTS

Hereinafter, embodiments of a blood pressure measurement apparatus, method, and program according to this invention will be described with reference to the drawings. Note that in the following embodiments, portions that have the same reference signs perform similar operations, and redundant descriptions will not be given for such portions.

### First Embodiment

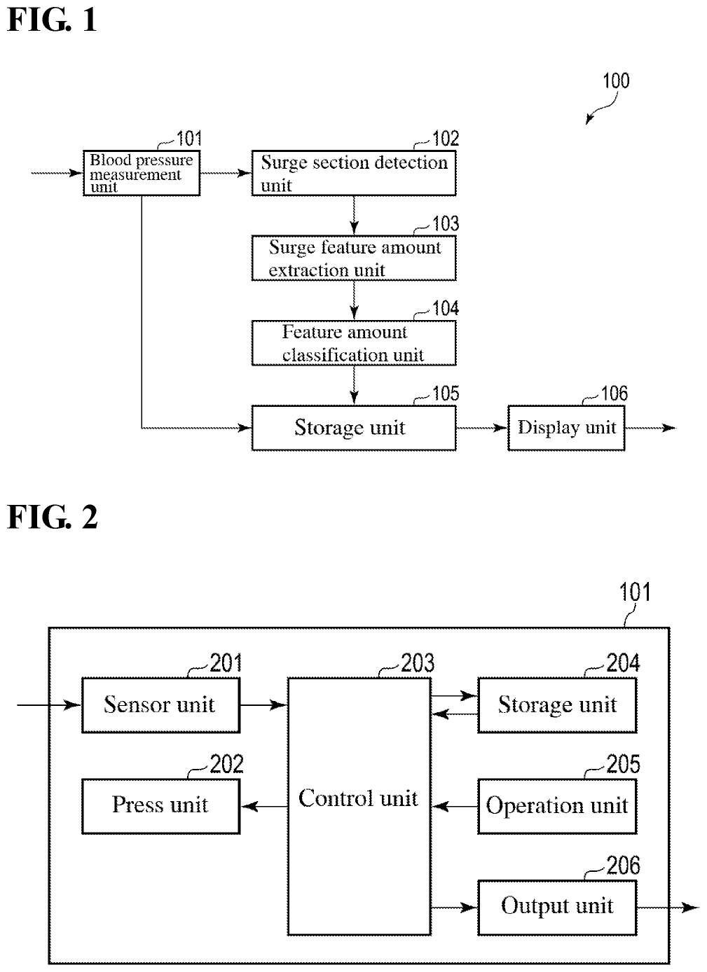

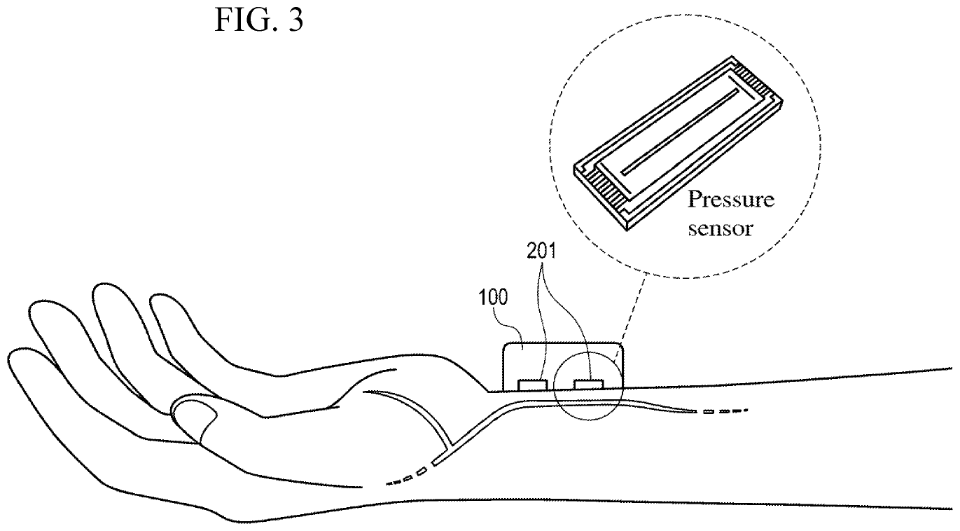

A blood pressure measurement apparatus 100 according to a first embodiment will be described below with reference to FIGS. 1 to 5. FIG. 1 is a functional block diagram of the blood pressure measurement apparatus 100, which includes a blood pressure measurement unit 101 that performs temporally continuous measurement, a surge section detection unit 102, a surge feature amount extraction unit 103, a feature amount classification unit 104, a storage unit 105, and a display unit 106. FIG. 2 is a functional block diagram of the blood pressure measurement unit 101, which can perform temporally continuous blood pressure measurement for each heartbeat using a tonometry method. FIG. 3 is an illustrative diagram showing a state in which the blood pressure measurement apparatus 100 is attached to a wrist, and shows a schematic transparent view in which the palm of the hand is viewed from the side (the side-by-side direction of the fingers when the hand is open). FIG. 3 shows an example in which two rows of pressure sensors are arranged extending in a direction orthogonal to a radial artery. Although FIG. 3 shows a state in which the blood pressure measurement apparatus 100 has been placed on the palm side of the arm, the blood pressure measurement apparatus 100 is fixedly wrapped around the arm in the actual usage state.

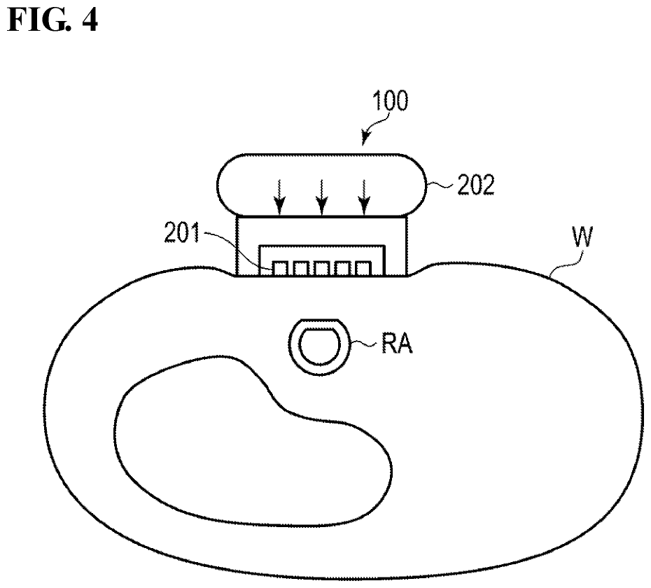

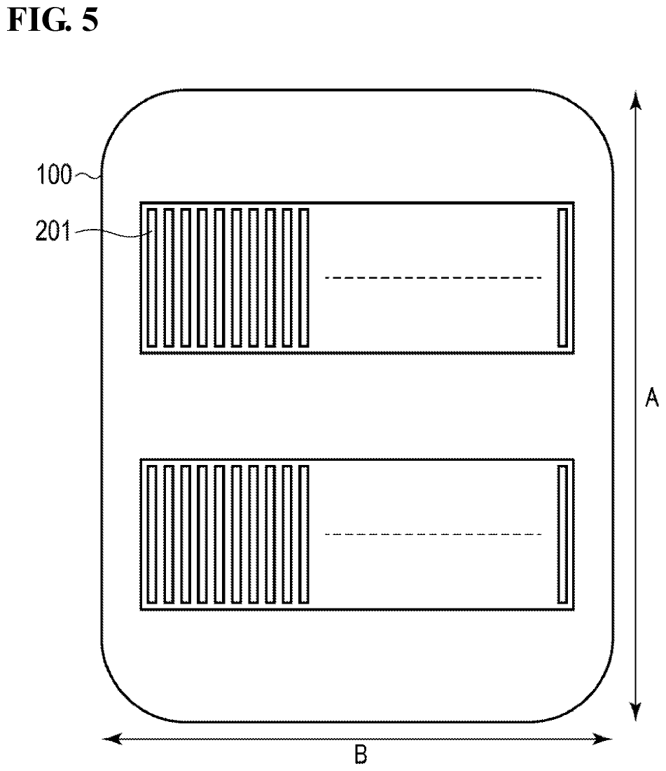

FIG. 4 is a cross-sectional view of the blood pressure measurement apparatus 100 and a wrist W, taken at a position of a sensor unit 201 in a state where the blood pressure measurement apparatus 100 is attached to the wrist. As shown in FIG. 4, the upper portion of a radial artery RA is being pressed by the blood pressure measurement apparatus 100 and has been flattened. FIG. 5 shows the blood pressure measurement apparatus 100 as viewed from the side in contact with the biological body, and in this figure, the sensors of the sensor unit 201 are arranged in two parallel rows on the contact surface. In each row of the sensor unit 201, a plurality of sensors are arranged side-by-side in a direction B that is orthogonal to a direction A in which the radial artery extends when the blood pressure measurement apparatus 100 is attached to the wrist W.

As shown in FIG. 1, the blood pressure measurement apparatus 100 includes the blood pressure measurement unit 101, the surge section detection unit 102, the surge feature amount extraction unit 103, the feature amount classification unit 104, the storage unit 105, and the display unit 106.

The blood pressure measurement apparatus 100 is loop-shaped for example, is wrapped around a wrist etc. like a bracelet, and measures a blood pressure based on biological information. As shown in FIGS. 2 and 3, the blood pressure measurement apparatus 100 is arranged such that the sensor unit 201 (e.g., pressure sensors) is located above the radial artery. It is also preferable that the blood pressure measurement apparatus 100 is arranged at a position corresponding to the height of the measurement subject's heart.

The blood pressure measurement unit 101 is for obtaining time-series data indicating a waveform of blood pressure values that continuously change in conjunction with heartbeats, and performs temporally continuous measurement of pressure pulse wave pressures of respective heartbeats with use of a tonometry method. Tonometry is a method of pressing an artery with a pressure sensor (e.g., a pressure pulse wave sensor), measuring pressure pulse waves, and then determining a blood pressure. Assuming that the artery is a circular artery having a uniform thickness, by taking the vascular wall into consideration, it is possible to derive a relational expression between the pressure inside the artery (blood pressure) and the pressure outside the artery (pressure pulse wave pressure) in accordance with Laplace's law, regardless of the flow of blood in the artery and the presence/absence of beating. With this relational expression, under the condition that the artery is being flattened at the pressed surface, by approximating the radius of the outer wall and the inner wall of the artery, it is possible to approximate that the pressure pulse wave pressure and the blood pressure are equivalent to each other. Accordingly, it is hereinafter assumed that the pressure pulse wave pressure is the same value as the blood pressure. As a result, the blood pressure measurement unit 101 can measure the blood pressure value of the subject biological body for each heartbeat.

The surge section detection unit 102 detects a surge section in continuous blood pressure value time-series data acquired from the blood pressure measurement unit 101. A surge is a sudden change in blood pressure, as previously mentioned. As a typical example here, a section that satisfies a surge condition is specified as a surge section. In other words, the condition for deeming a surge will not be strictly defined here. However, by merely replacing the surge condition described here with another condition, the blood pressure measurement apparatus 100 of the present embodiment can be applied to any condition for deeming a surge.

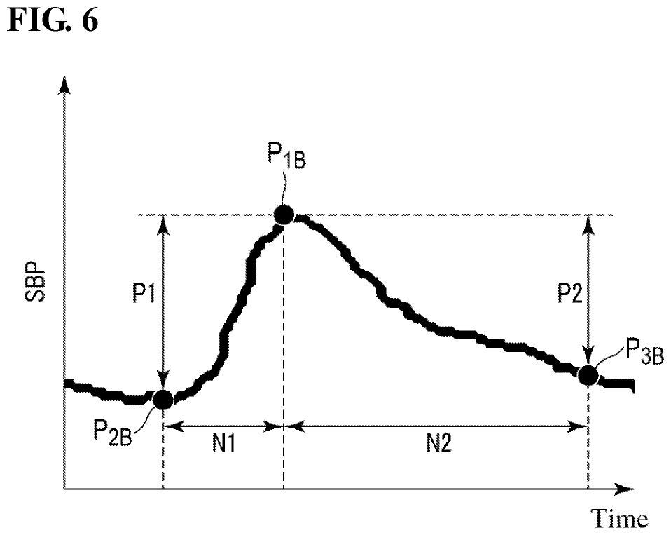

The following is a conceivable example of a typical condition for deeming that a portion of the blood pressure value time-series data corresponds to a surge. The following condition is expressed as a change in the value of the systolic blood pressure (SBP). However, the condition may be expressed as a change in any one or more of the systolic blood pressure, the diastolic blood pressure, the average blood pressure, and the pulse pressure. Note that actual SBP values do not form a smooth curve even when continuous values are acquired, and therefore, for easier use in subsequent processing, the blood pressure value time-series data is subjected to smoothing processing to obtain a curve that is continuous, smooth, and differentiable, for example. Here, it is assumed that the curve expressing the blood pressure value time-series data has been subjected to smoothing processing so as to be smooth and differentiable. The following description is given with reference to FIG. 6.

After the aforementioned smoothing processing is complete, as shown in FIG. 6, a peak point P_{1B} having a local maximum value is selected from the SBP time-series data. Normally, multiple peak points P_{1B} are discovered. Next, a search is performed to find a local minimum point P_{2B} having a local minimum value at a time before the peak point P_{1B}, and if the local minimum point P_{2B} is found, processing moves to the next condition. It is then judged whether or not a difference P1 between the blood pressure value at P_{1B} and the blood pressure value at P_{2B} is greater than a certain threshold value (e.g., 20 mmHg). If it is less than the threshold value, it is judged that it does not correspond to a surge. Next, it is judged whether a time difference N1 between the peak point P_{1B} and the local minimum point P_{2B} is greater than a certain time period (e.g., the time corresponding to five heartbeats), and if it is greater, it is judged that P_{2B} is a surge start point. Next, the point at which the derivative exceeds a certain value (e.g., −0.2 mmHg/s) at a future time after the peak point P_{1B} is obtained as a point P_{3B}. Next, it is judged whether a time difference N2 between the point P_{1B} and the point P_{3B} is greater than a certain time period (e.g., the time corresponding to seven heartbeats), and if it is greater, it is judged that P_{3B} is a surge end point. Here, if this time difference is greater than the time period, it is judged that the point P_{1B}, the point P_{2B}, and the point P_{3B} form a surge. In this case, the surge section detection unit 102 deems that the section from the point P_{2B} to the point P_{3B} is a surge section.

The surge feature amount extraction unit 103 extracts feature amounts of the detected surge. The surge feature amounts correspond to amounts that are related to the blood pressure values at the points P_{1B}, P_{2B}, and P_{3B} shown in FIG. 6 and also to time. The amounts shown in FIG. 6 are P1, P2, N1, and N2, for example. P1 denotes the difference between the blood pressure value at the surge peak point P_{1B} and the blood pressure value at the surge start point P_{2B}. P2 denotes the difference between the blood pressure value at the surge peak point P_{1B} and the blood pressure value at the surge end point P_{3B} (this difference will also be called the blood pressure change amount). N1 denotes the difference between the time at the surge peak point P_{1B} and the time at the surge start point P_{2B}, and will be called the rise time. N2 denotes the difference between the time at the surge peak point P_{1B} and the time at the surge end point P_{3B}, and will be called the fall time.

In the present embodiment, the surge feature amount extraction unit 103 extracts a rise time and a fall time for each surge. In other words, the surge feature amount extraction unit 103 extracts a rise time and a fall time for each surge section detected by the surge section detection unit 102.

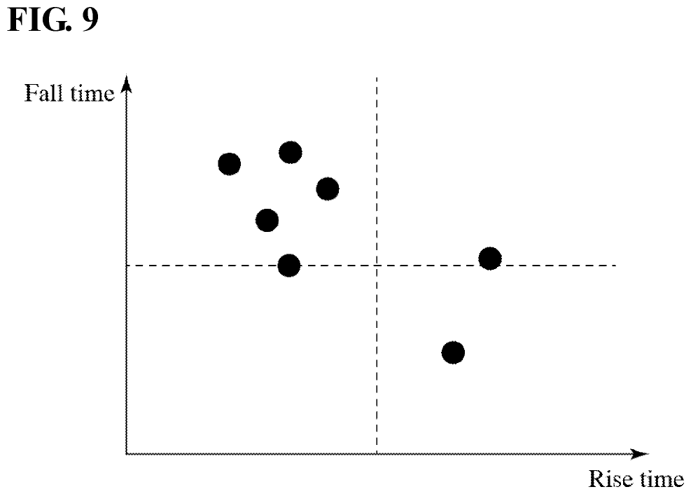

The feature amount classification unit 104 classifies surges based on the feature amounts extracted by the surge feature amount extraction unit 103. For example, the feature amount classification unit 104 maps the surges on a two-dimensional plane having the rise time and the fall time as axes, sets regions in the two-dimensional plane, and then classifies the surges. The classification performed by the feature amount classification unit 104 will be described later with reference to FIG. 9. Generally speaking, the feature amount classification unit 104 sets the number of dimensions to a number that is greater than or equal to 1 and less than or equal to the number of types of feature amounts, maps the surges in a space having the set number of dimensions with axes indicating feature amounts that correspond to the dimensions, and then classifies the surges.

The storage unit 105 stores classification results obtained by the feature amount classification unit 104. For example, the storage unit 105 stores classification results in association with corresponding biological bodies.

The display unit 106 displays classification results that are stored in the storage unit 105. For example, the display unit 106 may display classification results on an apparatus that is different from the blood pressure measurement apparatus 100, via a wireless unit.

Next, the blood pressure measurement unit 101 will be described with reference to FIG. 2.

The blood pressure measurement unit 101 includes the sensor unit 201, a press unit 202, a control unit 203, a storage unit 204, an operation unit 205, and an output unit 206. The sensor unit 201 performs temporally continuous pressure pulse wave detection. For example, the sensor unit 201 detects a pressure pulse wave for each heartbeat. The sensor unit 201 includes sensors that detect pressure, the sensor unit 201 is arranged on the palm side as shown in FIG. 3, and the sensors are normally arranged in two parallel rows that are side-by-side in the arm extending direction as shown in FIG. 3.

The sensor array includes a plurality of sensors in each row, and in each of the rows, a plurality of (e.g., 46) sensors are arranged side-by-side in a direction that intersects (is substantially orthogonal to) the arm extending direction. The press unit 202 is constituted by a pump, a valve, a pressure sensor, and an air bag, and when the air bag is inflated, the sensor portion of the sensor unit 201 is pressed against the wrist with an appropriate pressure, thus making it possible to raise the sensor sensitivity. Air is pumped into the air bag with use of the pump and the valve, the pressure sensor detects the pressure inside the air bag, and the control unit 203 monitors the detected pressure and performs control in order to adjust the pressure to an appropriate pressure. The control unit 203 performs overall control of the blood pressure measurement unit 101, receives pulse wave time-series data from the sensor unit 201, and converts the received data into blood pressure value time-series data, and stores the converted data in the storage unit 204.

The storage unit 204 stores blood pressure value time-series data, and provides requested data in response to a request from the control unit 203. The operation unit 205 accepts input from a user or the like via a keyboard, a mouse, a microphone, or the like, and also accepts instructions from an external server or the like wirelessly or via a wire. The output unit 206 receives blood pressure value time-series data from the storage unit 204 via the control unit 203, and transmits the received data to a device external to the blood pressure measurement unit 101.

As shown in FIGS. 3 and 4, the blood pressure measurement apparatus 100 is arranged on the palm side of the wrist, and the sensor unit 201 of the blood pressure measurement unit 101 is arranged at a position above the radial artery RA. As shown by arrows in FIG. 4, the press unit 202 presses the sensor unit 201 against the wrist W so as to flatten the radial artery RA. Note that although not shown in FIGS. 3 and 4, the blood pressure measurement apparatus 100 is loop-shaped and performs blood pressure measurement when wrapped around the wrist etc. like a bracelet.

Next, the sensor unit 201 of the blood pressure measurement apparatus 100 will be described with reference to FIG. 5. FIG. 5 shows the surface of the sensor unit 201 that comes into contact with the wrist W. As shown in FIG. 5, the sensor unit 201 includes one or more (two in this example) sensor arrays, and each sensor array has a plurality of sensors that are arranged side-by-side in the direction B. The direction B intersects the direction A, which is the extending direction of the radial artery, in the state where the blood pressure measurement apparatus 100 is attached to the measurement subject. For example, the direction A and the direction B may be orthogonal to each other. Each sensor array includes 46 sensors (this is also referred to as having 46 channels). Note that the sensors are assigned channel numbers here. Also, the sensor arrangement is not limited to the example shown in FIG. 5.

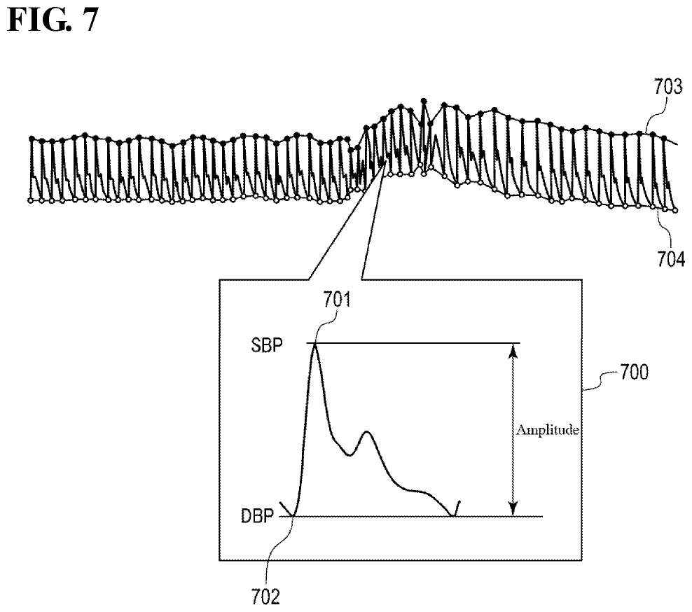

The sensors each measure pressure and generate pressure data. An element that converts pressure into an electrical signal can be used as the sensors. A pressure wave such as the wave shown in FIG. 7 is obtained as the pressure data. The pressure pulse wave measurement results are generated based on pressure data that has been output from one sensor that is adaptively selected from among the sensors (i.e., from one active channel). For each heartbeat, the maximum value in the pressure pulse waveform corresponds to the SBP, and the minimum value in the pressure pulse waveform corresponds to the diastolic blood pressure (DBP).

In addition to the pressure pulse wave measurement results, the blood pressure data can also include the pressure data that is output from each of the sensors. Note that rather than being generated in the blood pressure measurement unit 101, the pulse wave measurement results may be generated based on the pressure data by the control unit 203, which includes an information processing unit, in the blood pressure measurement apparatus 100. Also, the blood pressure measurement apparatus 100 may calculate the blood pressure value time-series data from the pressure pulse wave measurement results and output the calculated blood pressure value time-series data instead of the pulse wave measurement results.

Next, the blood pressure time-series data calculated from the pressure pulse wave measured by the blood pressure measurement unit 101 will be described with reference to FIG. 7. FIG. 7 shows blood pressure time-series data calculated from pressure pulse wave pressures obtained by measuring a pressure pulse wave pressure for each heartbeat. FIG. 7 also shows a blood pressure waveform 700 that is based on one of the pressure pulse waves. A blood pressure that is based on the pressure pulse wave is detected for each heartbeat in the form of a waveform as shown in FIG. 7, and the blood pressure that is based on the pressure pulse wave is detected continuously. The waveform 700 shown in FIG. 7 is a blood pressure waveform that is based on the pressure pulse wave of one heartbeat, a pressure value 701 corresponds to the SBP, and a pressure value 702 corresponds to the DBP. As shown by the time series of blood pressures corresponding to pressure pulse waves in FIG. 7, an SBP 703 and a DBP 704 of the blood pressure waveforms of heartbeats change over time.

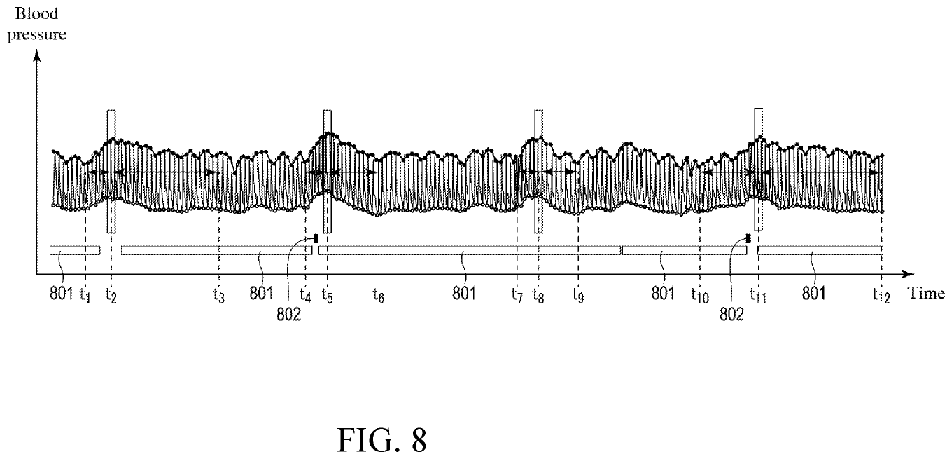

The following describes surge sections detected by the surge section detection unit 102 and the feature amounts extracted by the surge feature amount extraction unit 103 from the blood pressure value time-series data detected by the blood pressure measurement unit 101, with reference to FIG. 8.

The surge section detection unit 102 detects surge peak points. Peak points are discovered at times t₂, t₅, t₈, and t₁₁, and these peak points are shown in rectangular boxes in FIG. 8. The surge section detection unit 102 then detects surge start points. Surge start points are discovered at times t₁, t₄, t₇, and t₁₀. Also, surge end points are discovered at times t₃, t₆, t₉, and t₁₂. Reference signs 801 and 802 denote references, which are respectively a REM sleep state and an awakening reaction that have been detected by another detection apparatus. Note that one example of the surge start point discovery method is a method of finding a peak point, then finding the point corresponding to the minimum value at a time before the peak point, and then, if the difference between that minimum blood pressure value and the blood pressure value at the peak point is greater than or equal to a certain value, judging that the point having that minimum value is a surge start point. Another surge start point discovery method is a method of judging that a point is the start point if the point is temporally before the peak point and is lower than the peak value, and furthermore the difference between that point and the peak value is a certain value. Also, one example of the surge end point discovery method is a method of finding a surge start point, and then judging that a point is the end point if the point is temporally after the peak point and is lower than the peak value, and furthermore if the difference between that point and the peak value is a certain value.

In the present embodiment, the surge feature amount extraction unit 103 acquires a rise time and a fall time for each surge, and the feature amount classification unit 104 classifies feature amounts for each of them. Using the notation (rise time, fall time), the rise times and the fall times of the surges shown in the example in FIG. 8 are (|t1-t2|, |t2-t3|), (|t4-t5|, |t5-t6|), (|t7-t8|, |t8-t9|), and (|t10-t11|, |t11-t12|), where “| |” is an absolute value operator. Note that a surge index (SI) per unit of time and an AHI estimated from the SI may also be used as feature amounts. The SI and AHI estimation will be described later.

The following describes the classification performed by the feature amount classification unit 104 based on the rise times and the fall times of feature amounts extracted by the surge feature amount extraction unit 103 from the blood pressure value time-series data detected by the blood pressure measurement unit 101, with reference to FIG. 9.

FIG. 9 shows the result of the feature amount classification unit 104 mapping the rise times and the fall times on a two-dimensional plane. The points shown in FIG. 9 each correspond to one surge. In FIG. 9, the feature amount classification unit 104 has classified the rise times and the fall times as each being greater than or less than a certain threshold value, and thus in the two-dimensional map, the surges are classified into one of four regions and characterized as such.

It is said that the section corresponding to the surge rise time indicates autonomic nervous system coordination ability, and the section corresponding to the surge fall time indicates the blood pressure coordination ability. In other words, the longer the rise time, the stronger the autonomic nervous coordination ability is, and the shorter the fall time is, the stronger the blood pressure coordination ability is. By providing a threshold value on each axis and using them as indices, in FIG. 9, there are four classifications, namely a first quadrant in which the rise time and the fall time are both greater than the threshold values, a second quadrant in which the rise time is less than the threshold value and the fall time is greater than the threshold value, a third quadrant in which the rise time and the fall time are both less than the threshold values, and a fourth quadrant in which the rise time is greater than the threshold value and the fall time is less than the threshold value. Using the threshold values as boundaries, the feature amount classification unit 104 classifies the autonomic nervous coordination ability as weak or strong on the rise time axis, and classifies the blood pressure coordination ability as strong or weak on the fall time axis.

In the first quadrant, the autonomic nervous coordination ability is strong but the blood pressure coordination ability is weak; in the second quadrant, the autonomic nervous coordination ability is weak and the blood pressure coordination ability is also weak; in the third quadrant, the autonomic nervous coordination ability is weak but the blood pressure coordination ability is strong; and in the fourth quadrant, the autonomic nervous coordination ability is strong and the blood pressure coordination ability is also strong. If the points near the boundaries are excluded from the example in FIG. 9, zero events occur in the first quadrant, four events occur in the second quadrant, and one event occurs in the fourth quadrant, and furthermore, near the boundaries, one event occurs spanning the second quadrant and the third quadrant, and one event occurs spanning the first quadrant and the fourth quadrant.

In the case of this biological body, there is a high number of events in the second quadrant, and therefore the fact that the autonomic nervous coordination ability and the blood pressure coordination ability both tend to be weak can be immediately understood from the two-dimensional map in FIG. 9. Also, when examining the autonomic nervous coordination ability, five events are in weak regions, and two events are in strong regions. Furthermore, when examining the blood pressure coordination ability, excluding the boundary regions, four events are in weak regions, and one event is in a strong region. When examining the occurrence ratio, the autonomic nervous coordination ability is 5/2, and the blood pressure coordination ability is 4/1, and therefore it is understood that weakness of the blood pressure coordination ability is more likely to occur than weakness of the autonomic nervous coordination ability.

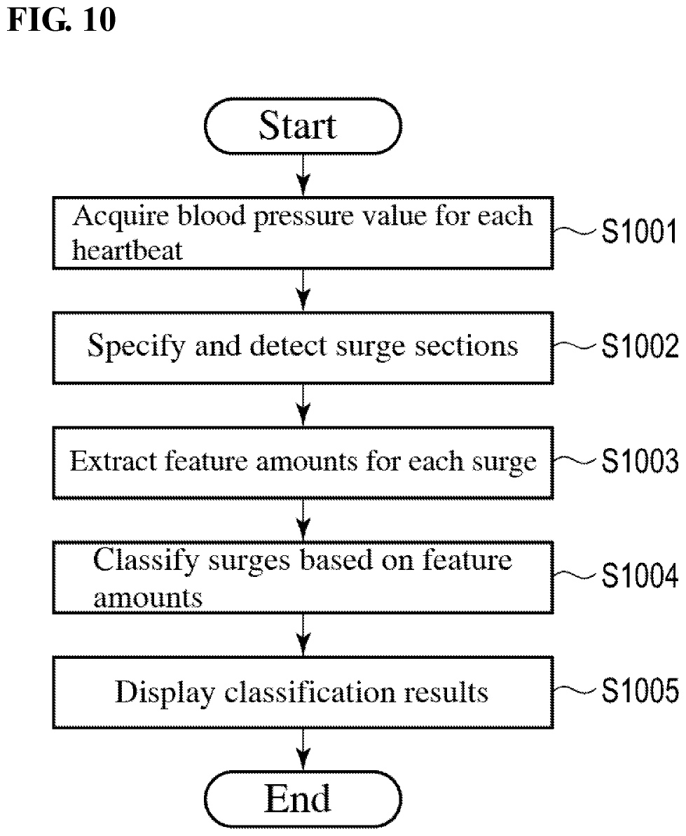

Next, an example of operations of the blood pressure measurement apparatus 100 will be described with reference to FIG. 10. FIG. 10 is a flowchart showing a typical example of operations of the blood pressure measurement apparatus 100.

The blood pressure measurement unit 101 acquires blood pressure value time-series data from the biological body, and passes the acquired data to the surge section detection unit 102 (step S1001). The blood pressure measurement unit 101 passes the time-series data to the storage unit 105, and the storage unit 105 successively stores the blood pressure value time-series data.

In step S1002, the surge section detection unit 102 detects surge sections in the blood pressure value time-series data, and passes information indicating the surge sections and the time-series data to the surge feature amount extraction unit 103.

In step S1003, the surge feature amount extraction unit 103 extracts surge feature amounts for each surge specified from the surge sections. In the example in FIG. 9, a rise time and a fall time are extracted as feature amounts in each surge section, but the feature amounts may be any type of feature amount that can be extracted from a surge.

In step S1004, the feature amount classification unit 104 classifies the surges based on the extracted feature amounts. The feature amount classification unit 104 maps the surges that correspond to the feature amounts in a multi-dimensional space that corresponds to the number of types of feature amounts that are being focused on. In this case, the number of dimensions is greater than or equal to 1 and less than or equal to the number of types of feature amounts. In the example in FIG. 9, there are two types of feature amounts, namely the rise time and the fall time, and therefore points corresponding to surges specified by two feature amounts are mapped in a two-dimensional space. The data obtained by the feature amount classification unit 104 mapping the surges in a multi-dimensional space is stored in the storage unit 105.

In step S1005, the display unit 106 receives the multi-dimensional space map data stored in the storage unit 105, and displays the received data. For example, the display in FIG. 9 is shown. If, for each biological body, feature amounts expressing types of coordination performance are mapped in a multi-dimensional space as shown in FIG. 9, it is easy to see where a problem lies, and such information can be useful when a doctor makes a treatment plan for example.

According to the blood pressure measurement apparatus of the first embodiment described above, one or more surge sections each including one surge are detected from blood pressure value time-series data, one or more feature amounts are extracted for each surge, and the surges are classified based on the feature amount, thus making it possible to obtain features related to surges in the biological body from which the blood pressure values were acquired. A weakness of the biological body can be easily screened based on these features, and if the biological body is determined to have an illness, it is possible to judge whether or not the biological body needs medicine or whether there is a need for more thorough examination.

Also, by mapping the surges in a multi-dimensional space having the feature amounts as axes, the features of the surges are mapped in a space, it is easy to classify features according to the regions in the space, and surge features can be understood immediately. Accordingly, features of a biological body that are causing surges can also be understood, and such information can be useful when a doctor makes a treatment plan for example.

### Second Embodiment

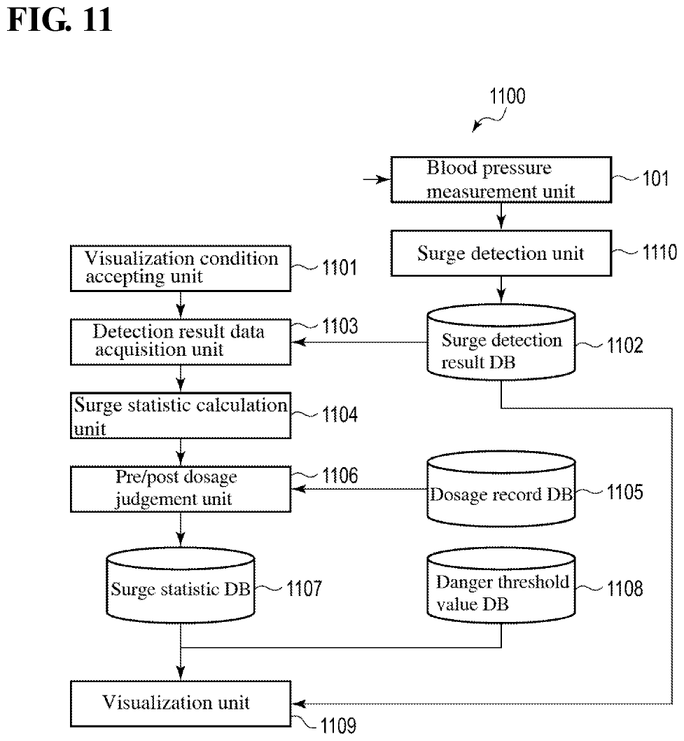

A blood pressure measurement apparatus according to a second embodiment will be described below with reference to FIG. 11. FIG. 11 is a block diagram showing the blood pressure measurement apparatus of the second embodiment. The blood pressure measurement apparatus of the second embodiment is different from the blood pressure measurement apparatus of the first embodiment in that a surge statistic is also calculated. Furthermore, the blood pressure measurement apparatus of the second embodiment also takes a dosage record into consideration and changes a visualization accordingly.

A blood pressure measurement apparatus 1100 of the present embodiment includes a blood pressure measurement unit 101, a surge detection unit 1110, a visualization condition accepting unit 1101, a surge detection result database (also called a “DB”) 1102, a detection result data acquisition unit 1103, a surge statistic calculation unit 1104, a dosage record DB 1105, a pre/post dosage judgment unit 1106, a surge statistic DB 1107, a danger threshold value DB 1108, and a visualization unit 1109.

The surge detection unit 1110 is a combination of the surge section detection unit 102 and the surge feature amount extraction unit 103. In other words, the surge section detection unit 1110 detects a surge section in continuous blood pressure value time-series data acquired from the blood pressure measurement unit 101. The surge detection unit 1110 then extracts feature amounts of the detected surge. The surge feature amounts correspond to amounts that are related to the blood pressure values at the points P_{1B}, P_{2B}, and P_{3B} shown in FIG. 6 and also to time. Specifically, the surge feature amounts are a blood pressure change amount, a rise time, and a fall time. The blood pressure change amount is the amount of change in the blood pressure over the rise time, and is said to indicate the extent of the burden on the respiratory system. The higher the blood pressure change amount is, the higher the burden is. Note that the rise time and the fall time are the same as in the description given in the first embodiment.

The visualization condition accepting unit 1101 accepts a visualization condition that indicates which content is to be visualized (e.g., be displayed on a display screen), specifically which of the surge feature amounts are to be variables.

Examples of aspects of visualization include a one-dimensional plot having the surge index (also called “SI”) as a variable, and a two-dimensional plot having the rise time and the fall time as variables. The surge index referred to here is the number of surges per hour. One example of the display content is a summary of results obtained from one user in one instance. One instance refers to the time from the start to the end of continuous blood pressure measurement of the subject. Here, one instance is envisioned to be one night. In the example described in the present embodiment, the surge detection unit 1110 accepts an indication of one of the following four types of content: Example 1 corresponding to plotting a summary of results of one person in one instance, Example 2 corresponding to plotting a summary of results of one person in multiple instances, Example 3 corresponding to plotting all results of one person in one instance, and Example 4 corresponding to plotting a summary of results of multiple subjects in one instance.

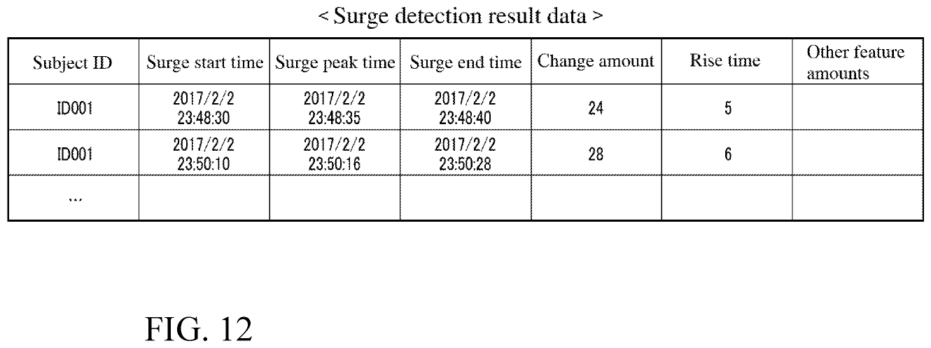

The surge detection result DB 1102 records data indicating feature points and feature amounts detected by the blood pressure measurement unit 101 and the surge detection unit 1110. In the present embodiment, the data to be recorded includes data from an individual in one instance, data from an individual in multiple instances, and data from all subjects in one instance. The feature points and the feature amounts are similar to those described in the first embodiment. An example of the data stored in the surge detection result DB 1102 will be described later with reference to FIG. 12.

The detection result data acquisition unit 1103 acquires detection result data from the surge detection result DB 1102 based on a visualization condition from the visualization condition accepting unit 1101.

The surge statistic calculation unit 1104 calculates surge statistics from the detection result data acquired by the detection result data acquisition unit 1103. Examples of the surge statistic include a surge index, an average rise time, an average fall time, an average blood pressure change amount, and standard deviations (also called “SDs”) thereof.

The dosage record DB 1105 records data indicating which medicines have been given and at what time for each subject. The dosage record DB 1105 records information regarding the dosage start date/time and the type of medicine. An example of the data stored in the dosage record DB 1105 will be described later with reference to FIG. 15.

The pre/post dosage judgment unit 1106 judges, with reference to the dosage record DB 1105, whether or not a surge statistic calculated by the surge statistic calculation unit 1104 (i.e., a detected surge) corresponds to a pre-dosage time or a post-dosage time. Here, a pre-dosage time refers to a time before the start of a dosage, and refers to a state in which medicine has not been given, whereas a post-dosage time refers to a time after the start of a dosage, and refers to a state in which medicine is being continuously given.

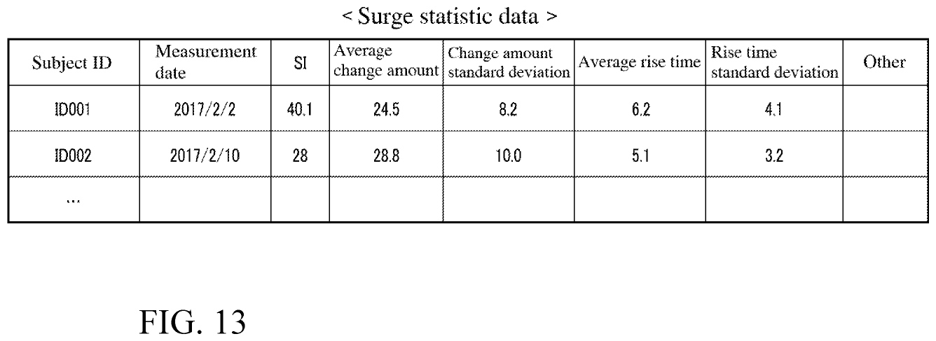

The surge statistic DB 1107 records the surge statistic calculated by the surge statistic calculation unit 1104 for each subject. An example of the data stored in the surge statistic DB 1107 will be described later with reference to FIG. 13.

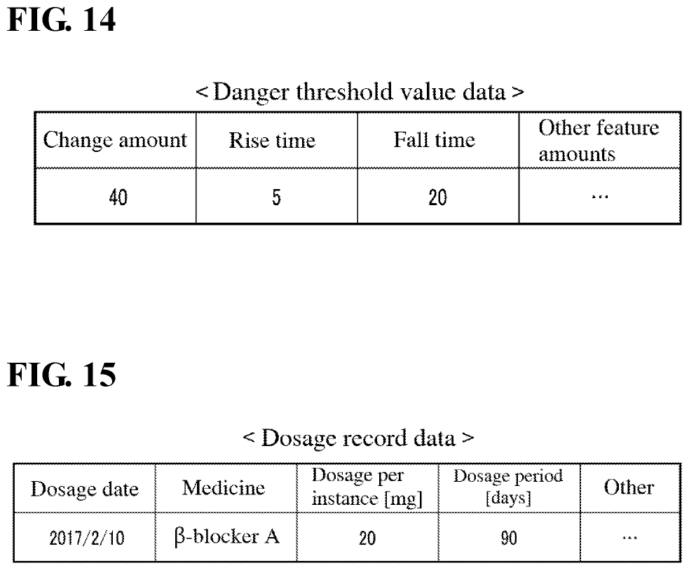

Threshold values that correspond to surge statistics and are associated with danger are stored in advance in the danger threshold value DB 1108. Also, in the case where the detection result data acquisition unit 1103 has acquired data for multiple instances, the danger threshold value DB 1108 may set and store threshold values based on how much a surge statistic of a certain subject deviates from the average. For example, in the case where the threshold value corresponds to a difference of a standard deviation of 1 from the average, an affirmative judgement is made if the standard deviation from the average value is greater than or equal to 1. An example of the data stored in the danger threshold value DB 1108 will be described later with reference to FIG. 14. Comparison of the threshold values and the surge statistics may be performed by the visualization unit 1109 for example.

The visualization unit 1109 visualizes the surge feature amounts using a one or more dimensional plot having variables on one or more axes. Different colors are used to enable identifying pre-dosage and post-dosage times for each subject and each medicine.

Next, examples of the data stored in the surge detection result DB 1102, the surge statistic DB 1107, the danger threshold value DB 1108, and the dosage record DB 1105 will be described with reference to FIGS. 12, 13, 14, and 15.

As shown in the example in FIG. 12, the surge detection result data stored in the surge detection result DB 1102 includes a subject ID, a surge start time, a surge peak time, a surge end time, a change amount, and a rise time.

As shown in the example in FIG. 13, the surge statistic data stored in the surge statistic DB 1107 includes a subject ID, a measurement date, an SI, an average change amount, a change amount standard deviation, an average rise time, and a rise time standard deviation. The surge statistic data includes one row for one person in one instance in Example 1, and includes all data for one person in one instance in Example 2. In Example 2, if there are multiple instances for one person, the data includes one row for each instance of measurement. In Example 4, there are a plurality of people in one instance, and the data includes one row for each subject.

As shown in the example in FIG. 14, the danger threshold value data stored in the danger threshold value DB 1108 includes a change amount, a rise time, and a fall time. It is envisioned that there is only one type of threshold value in the example described below, but there may be a plurality of threshold values corresponding to levels of danger.

As shown in the example in FIG. 15, the dosage record data stored in the dosage record DB 1105 includes a dosage date, a medicine name, a dosage amount per instance, and a dosage period. Only one type of medicine is envisioned in the examples described below, but information for multiple types of medicine may be stored.

### EXAMPLE 1

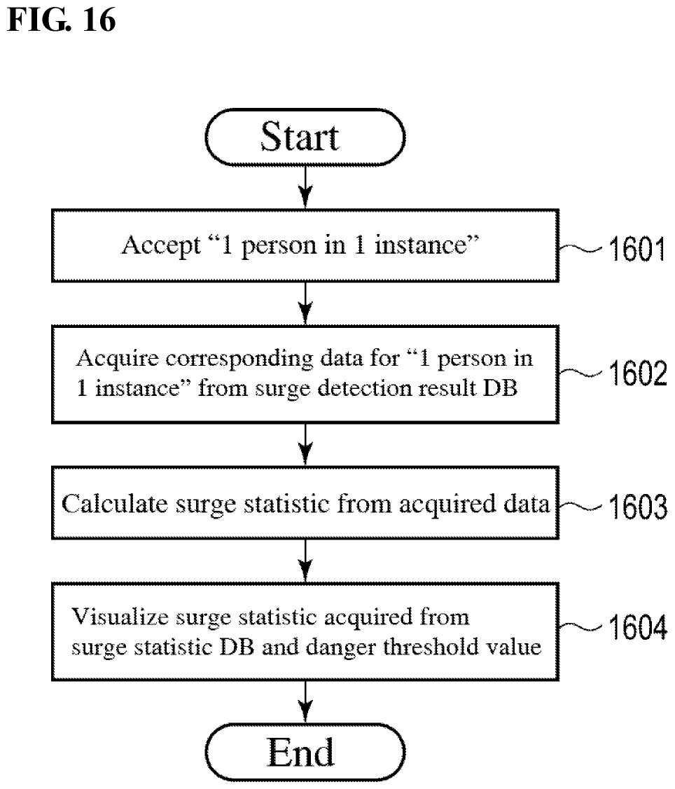

Next, Example 1 will be described with reference to FIGS. 16, 17, and 18. Example 1 is a case of plotting a summary of results for one person in one instance. The visualization condition accepting unit 1101 accepts an indication to the effect of “one person in one instance”, and transmits, to the detection result data acquisition unit 1103, an indication for visualizing surge detection performed in one night for a certain subject (step S1601). The detection result data acquisition unit 1103 acquires data corresponding to one person in one instance from the surge detection result DB 1102 (step S1602).

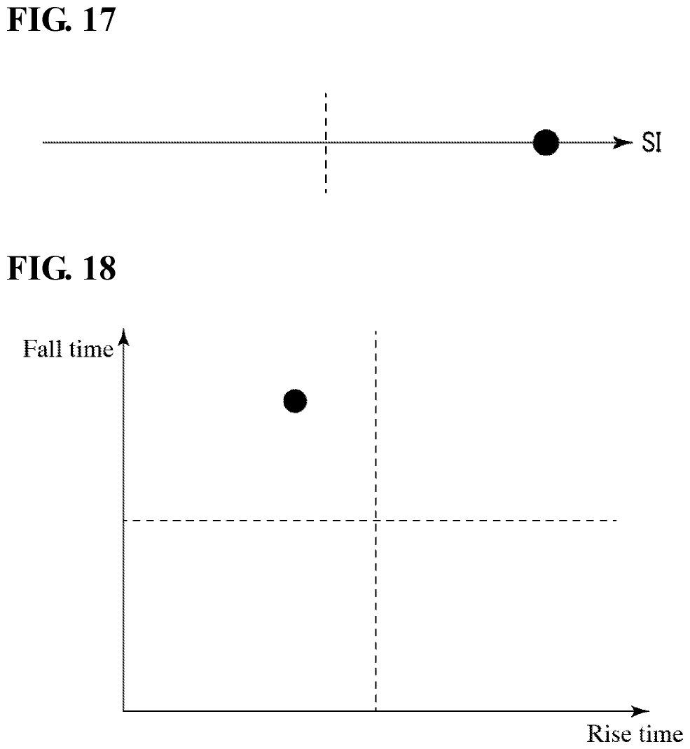

The surge statistic calculation unit 1104 calculates surge statistics from the surge detection result data (step S1603). For example, the surge statistic calculation unit 1104 calculates statistics regarding the surge index, the fall time, and the rise time shown in FIGS. 17 and 18, and records the calculated statistics in the surge statistic DB 1107. In the case of one person in one instance, the data corresponds to only one night, and therefore the pre/post dosage judgment unit 1106 does not make a judgment.

The visualization unit 1109 visualizes the surge statistics acquired from the surge statistic DB 1107 and danger threshold values acquired from the danger threshold value DB 1108 (step S1604). For example, the visualization unit 1109 obtains plots as shown in FIGS. 17 and 18. Although one-dimensional and two-dimensional plots are shown here, they may be combined into a three-dimensional plot. Alternatively, the blood pressure change amount may also be included to obtain a multi-dimensional plot.

### EXAMPLE 2

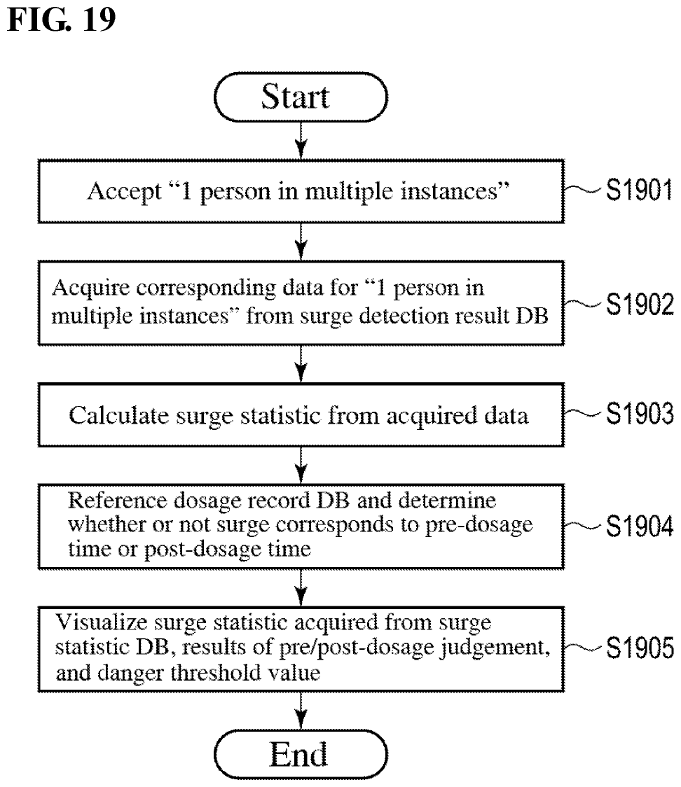

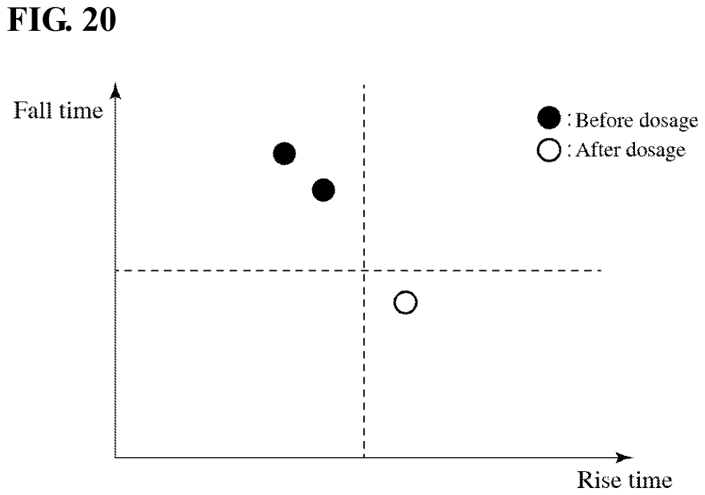

Next, Example 2 will be described with reference to FIGS. 19 and 20. Example 2 is a case of plotting a summary of results for one person in multiple instances.

The visualization condition accepting unit 1101 receives an indication to the effect of “one person in multiple instances”, and transmits, to the detection result data acquisition unit 1103, an indication for visualizing surge detection performed over multiple nights for a certain subject (step S1901). The detection result data acquisition unit 1103 acquires data corresponding to one person in multiple instances from the surge detection result DB 1102 (step S1902).

The surge statistic calculation unit 1104 calculates surge statistics from the surge detection result data (step S1903). The pre/post dosage judgment unit 1106 judges, with reference to the dosage record DB 1105, whether or not the surge statistic calculated by the surge statistic calculation unit 1104 corresponds to a pre-dosage time or a post-dosage time, and records the surge statistic along with judgement result in the surge statistic DB 1107 (step S1904).

The visualization unit 1109 acquires the surge statistics and the results of pre/post-dosage judgement from the surge statistic DB 1107, and acquires danger threshold values from the danger threshold value DB 1108, and visualizes the acquired data (step S1905). FIG. 20 shows an example of the visualization obtained by the visualization unit 1109. According to FIG. 20, it is possible to make a comparison between pre-dosage times and post-dosage times, and it can be seen that the fall time is shorter after dosage than before dosage. The shorter the fall time is, the higher the possibility is that the blood pressure adjustment force is excellent, and therefore it can be understood that there is a high possibility that the medicine had an effect. In this way, based on the results for one person in multiple instances, it is possible to understand state changes and the effect of medicine since the start of treatment.

### EXAMPLE 3

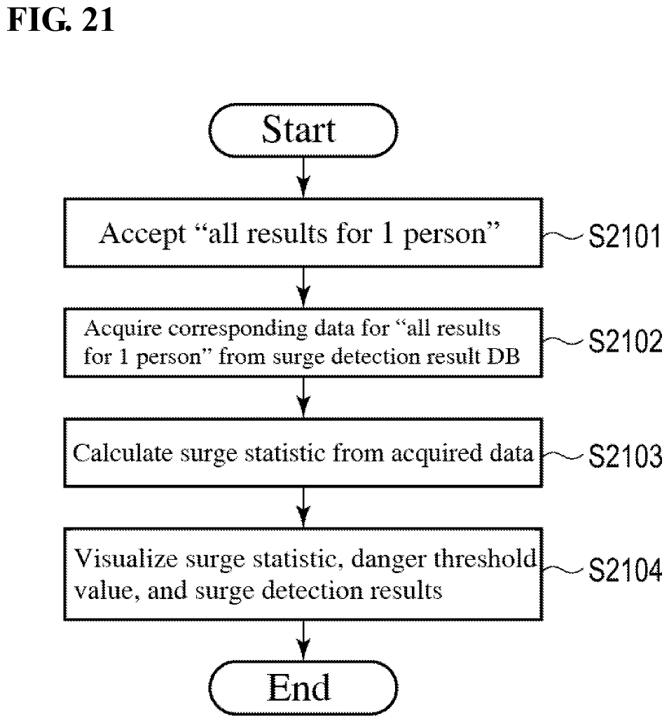

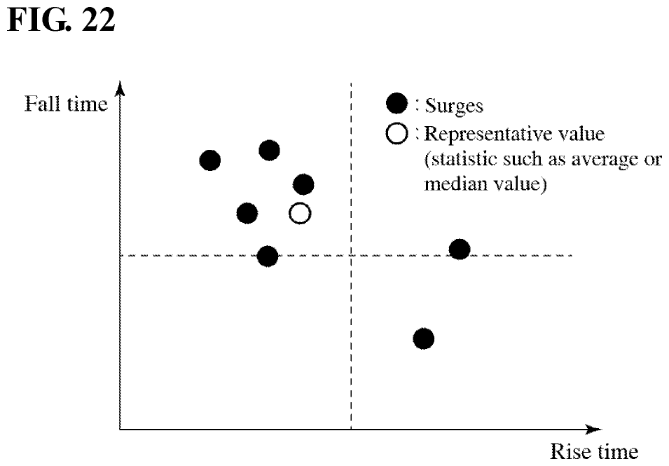

Next, Example 3 will be described with reference to FIGS. 21 and 22. Example 3 is a case of plotting a summary of all results for one person in one instance.

The term “all results” includes not only statistics, but also the feature amounts of each surge.

The visualization condition accepting unit 1101 accepts an indication to the effect of “all results for one person in one instance”, and transmits, to the detection result data acquisition unit 1103, an indication for visualizing not only the statistics but also the feature amounts of each surge obtained in surge detection performed in one night for a certain subject (step S2101). The detection result data acquisition unit 1103 acquires data corresponding to one person in one instance from the surge detection result DB 1102 (step S2102).

The surge statistic calculation unit 1104 calculates surge statistics from the acquired surge detection result data, and records the calculated surge statistics in the surge statistic DB 1107 (step S2103). The visualization unit 1109 acquires and visualizes the surge statistics acquired from the surge statistic DB 1107, danger threshold values acquired from the danger threshold value DB 1108, and the feature amounts of the surges acquired from the surge detection result DB 1102 (step S2104). For example, the visualization unit 1109 plots the data as shown in FIG. 22, and displays the feature amounts of the surges (here, rise times and fall times) and a representative value that is a surge statistic (e.g., an average or a median value). Note that similarly to Example 1, in the case of one person in one instance, the data corresponds to only one night, and therefore the pre/post dosage judgment unit 1106 does not make a judgment.

According to Example 3, all surges in one night are plotted, thus making it possible to check not only statistics, but also the distribution of the surges themselves. As a result, it is possible to find out whether or not change in the surge feature amounts conforms to a normal distribution.

### EXAMPLE 4

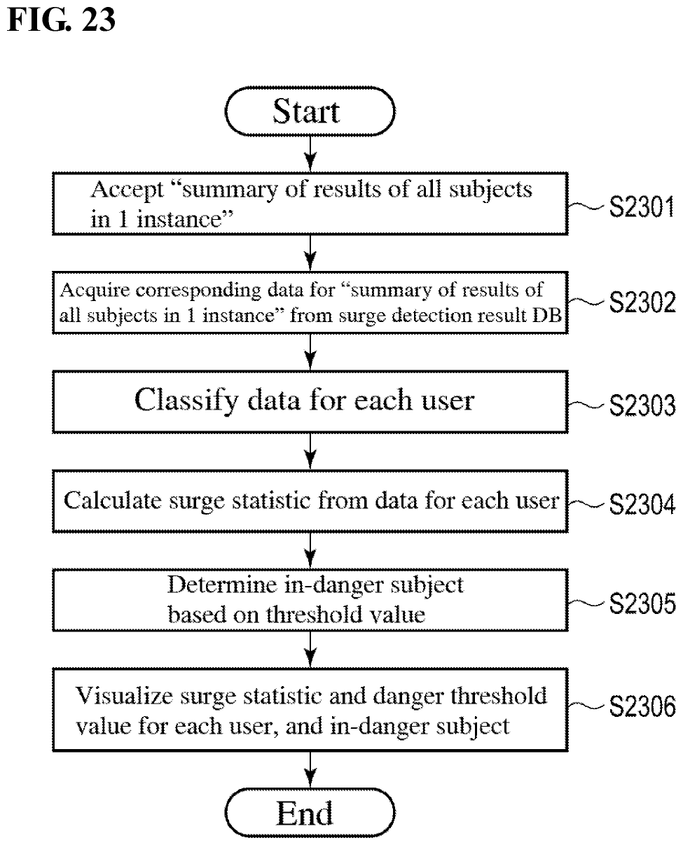

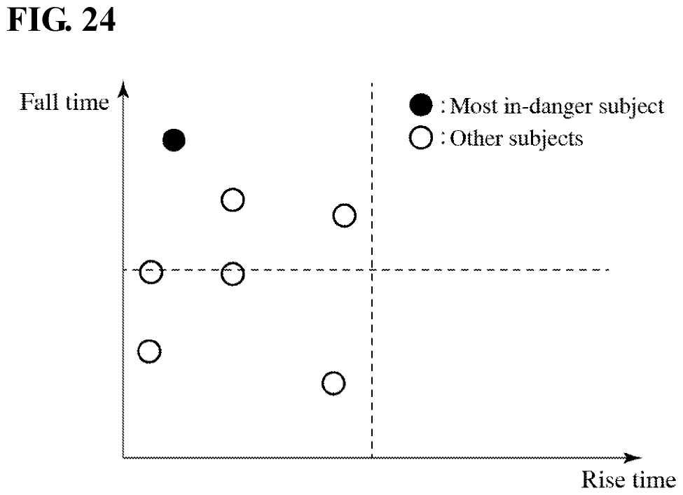

Next, Example 4 will be described with reference to FIGS. 23 and 24. Example 4 is a case of plotting a summary of results for multiple subjects in one instance. This makes it possible to estimate the danger level for each subject, and also facilitates comparison with other people. It is also possible to check overall trends for the subjects.

The visualization condition accepting unit 1101 accepts an indication to the effect of “results of all subjects in one instance”, and transmits, to the detection result data acquisition unit 1103, an indication for visualizing statistics obtained in surge detection performed in one night for all subjects (step S2301). The detection result data acquisition unit 1103 acquires data corresponding to all subjects in one instance from the surge detection result DB 1102 (step S2302). Furthermore, the detection result data acquisition unit 1103 classifies the data for each user, and transmits the surge detection results for each user to the surge statistic calculation unit 1104 (step S2303). The surge statistic calculation unit 1104 receives the surge detection results and calculates surge statistics for each user (step S2304). When there are multiple subjects, multiple subject IDs are recorded in the surge detection result DB 1102.

Based on the surge statistics of the users and threshold values acquired from the danger threshold value DB 1108, the visualization unit 1109 determines a subject for which the level of danger is high, or in other words, specifies the subject who has the highest level of danger (is in the most danger) for example (step S2305). The visualization unit 1109 then visualizes the subjects and the subject having the highest level of danger as shown in FIG. 24 (step S2306). The visualization unit 1109 visualizes the surges according to the levels of danger, sets a danger reference that corresponds to a threshold value, and enables identifying whether a surge is at a dangerous level.

According to the blood pressure measurement apparatus of the second embodiment described above, it is possible to achieve the effects of the first embodiment, and to also acquire surge statistics and give consideration to dosage status data as well, thus making it possible to judge state changes from the start of treatment and the efficacy of medicine on patients. Data is plotted for multiple patients, thus making it possible to visualize the level of danger for each patient. It is therefore possible to find out overall trends for the patients.

### Third Embodiment

A blood pressure measurement apparatus 2500 according to a third embodiment is the blood pressure measurement apparatus 1100 according to the second embodiment, which additionally includes an SI (Surge Index) calculation unit 2501, an Apnea Hypopnea Index (AHI) calculation unit 2502, and an AHI-SI correlation DB 2503. The units other than these three additional units, as well as the operations thereof, are similar to the corresponding units in the second embodiment. In other words, the SI and estimated AHI may be used as feature amounts when performing visualization. Note that the AHI is closely related to SAS symptoms, and it can be said that the higher the AHI is, the more severe the SAS is.

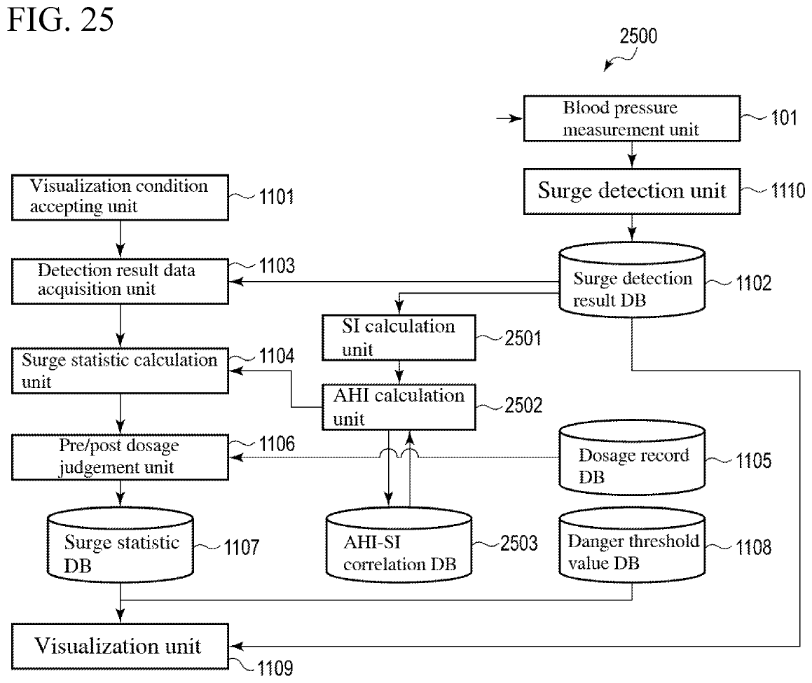

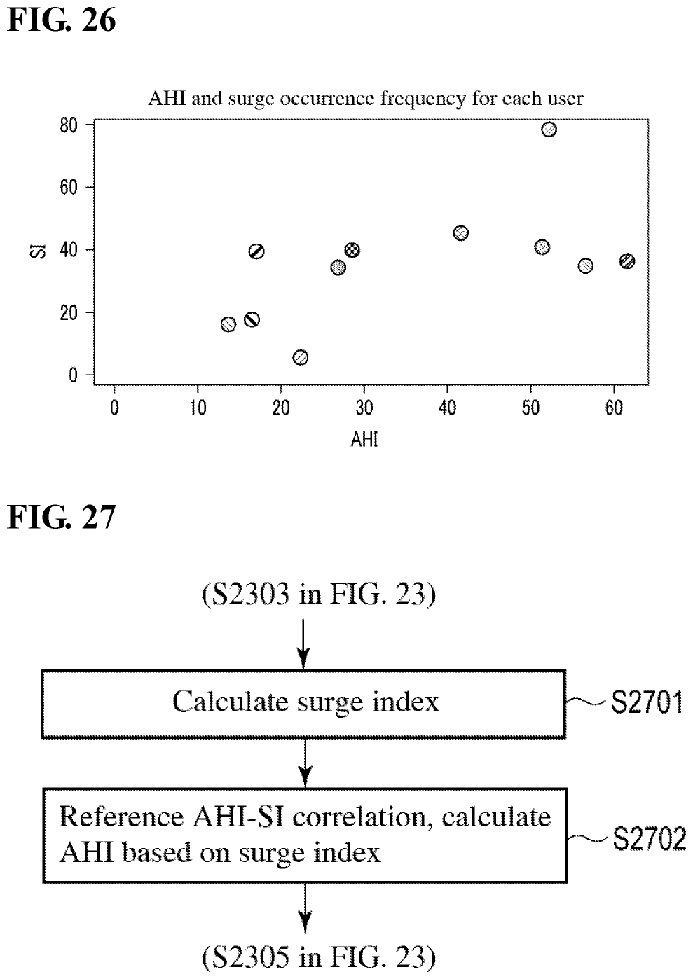

Next, the blood pressure measurement apparatus 2500 will be described with reference to FIGS. 25 and 26. FIG. 25 is a block diagram showing the blood pressure measurement apparatus 2500. FIG. 26 is a diagram showing the results of measuring the AHI and the SI for each user. The AHI is measured with a different method from that of the apparatus of the present embodiment, such as a PSG (Polysomnography).

For each user, the SI calculation unit 2501 obtains a surge index, which is the frequency of blood pressure surges per hour (e.g., the number of occurrences of a blood pressure surge per hour), from data in the surge detection result DB 1102.

The AHI calculation unit 2502 obtains the AHI based on the surge index from the SI calculation unit 2501 and correlation data stored in the AHI-SI correlation DB 2503. AHI refers to the Apnea Hypopnea Index and indicates the total number of apnea and hypopnea incidents per hour of sleep. Note that hypopnea refers to a state where the arterial blood vessel oxygen saturation level (SpO2) has decreased by 3-4% or more, or a state that is accompanied by awakening.

The AHI-SI correlation DB 2503 stores data that indicates a correlation between the AHI and the SI. The AHI-SI correlation DB 2503 stores, in advance, data that indicates a correlation relationship derived from the results of plotting AHI and SI data for each user as shown in FIG. 26.

Data such as that shown in FIG. 26 is collected, and a correlation between the AHI and the SI is calculated based on the collected data. The example in FIG. 26 shows results in which the correlation coefficient is 0.59, and there is an intermediate level of correlation. Accordingly, there is a correlation between the AHI and the SI, and therefore based on the data shown in FIG. 26, it is possible to use only the SI as a seriousness index for stratification, and it is possible to also estimate the AHI.

Next, operations of the blood pressure measurement apparatus 2500 in FIG. 25 will be described with reference to FIG. 27.

After branching from step S2303 in FIG. 23, processing moves through steps S2701 and S2702, and then merges with the operations in Example 4 of the second embodiment in step S2305 in FIG. 23.

In step S2701, the SI calculation unit 2501 references the blood pressure surge detection result data that was detected and stored in step S2303, and calculates a surge index. Next, in step S2702, the AHI-SI correlation DB 2503, which stores correlation information regarding the AHI and the surge index, is referenced to calculate the AHI based on the surge index obtained in step S2701.

According to the third embodiment described above, it is possible to find out the correlation between the surge frequency and the AHI, and this enables determining the AHI by merely measuring blood pressure surges, thus making it possible to easily find out the SAS seriousness level.

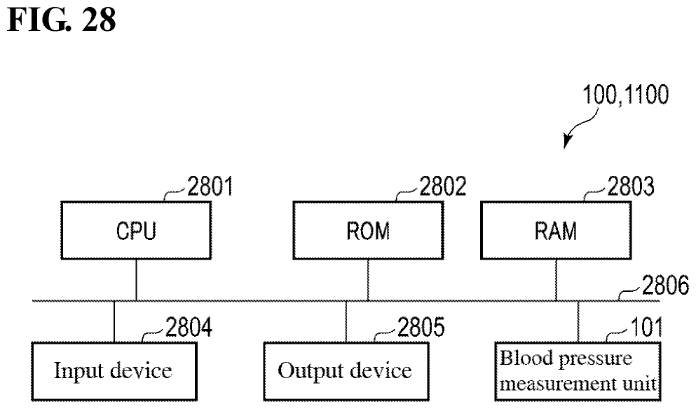

Next, an example of the hardware configuration of the blood pressure measurement apparatus 100 (1100) will be described with reference to FIG. 28.

The blood pressure measurement apparatus 100 (1100) includes a CPU 2801, a ROM 2802, a RAM 2803, an input device 2804, an output device 2805, and the blood pressure measurement unit 101, and these constituent elements are connected to each other via a bus system 2806. The above-described functions of the blood pressure measurement apparatus 100 (1100) can be realized by the CPU 2801 reading out a program stored in a computer-readable recording medium (ROM 2802) and executing the program. The CPU 2801 uses the RAM 2803 as a work memory. Alternatively, a configuration is possible in which an auxiliary storage device (not shown) includes a hard disk drive (HDD) or a solid state drive (SDD) for example, and is used as the storage unit 105, the surge detection result DB 1102, the dosage record DB 1105, the surge statistic DB 1107, the danger threshold value DB 1108, and the AHI-SI correlation DB 2503, and furthermore stores the aforementioned program.

The input device 2804 includes a keyboard, a mouse, a microphone, and the like, and accepts operations from a user. The input device 2804 includes operation button for causing the blood pressure measurement unit 101 to start measurement, an operation button for performing calibration, and an operation button for starting and stopping communication. The output device 2805 includes a display device such as a liquid crystal display device, and a speaker, for example. The blood pressure measurement unit 101 exchanges signals with another computer via a communication device for example, and receives measured data from a blood pressure measurement apparatus for example. The communication device often uses a communication method capable of exchanging data over a short distance, such as short-range wireless communication, specific examples of which include Bluetooth (registered trademark), TransferJet (registered trademark), Zigbee (registered trademark), and IrDA (registered trademark).

Also, in the above-described embodiment, the blood pressure measurement unit 101 detects the pressure pulse wave of a radial artery that passes under the measurement site that is the left wrist or the like (tonometry method). However, there is no limitation to this. The blood pressure measurement unit 101 may detect a change in impedance as the pulse wave of the radial artery that passes under the measurement site that is the left wrist or the like (impedance method). The blood pressure measurement unit 101 may include a light emitting element that emits light toward an artery that passes through a corresponding portion of the measurement site, and a light receiving element that receives reflected light (or transmitted light), and detect a change in volume as the pulse wave of the artery (photoelectric sensing method). Also, the blood pressure measurement unit 101 may include a piezoelectric sensor that is brought into contact with the measurement site, and detect a change in electrical resistance as distortion caused by pressure of an artery that passes through a corresponding portion of the measurement site (piezoelectric method). Furthermore, the blood pressure measurement unit 101 may include a transmission element that transmits radio waves (transmission waves) toward an artery that passes through a corresponding portion of the measurement site, and a reception element that receives reflected waves, and detect a shift in the phases of the transmission waves and the reflected wave as a change in the distance between the artery and the sensor caused by artery pulse waves (radio wave emission method). Note that another method may be applied as long as it can observe a physical amount from which the blood pressure can be calculated.

Also, a configuration is possible in which the ROM 2802 or the auxiliary storage device stores a program for executing operations performed by the surge section detection unit 102, the surge feature amount extraction unit 103, the feature amount classification unit 104, the visualization condition accepting unit 1101, the detection result data acquisition unit 1103, the surge statistic calculation unit 1104, the pre/post dosage judgment unit 1106, and the visualization unit 1109 that are described above, and the CPU 2801 executes that program. Alternatively, the program may be stored in a sever or the like that is different from the blood pressure measurement apparatus 100, and a CPU of the server or the like may execute the program. In this case, pressure pulse wave time-series data (or blood pressure value time-series data) measured by the blood pressure measurement unit 101 can be transmitted to the server and subjected to processing in the server, and a reliability can be obtained. In this case, processing is performed by the server, and therefore the processing speed can possibly increase. Furthermore, the constituent portions of the surge section detection unit 102, the surge feature amount extraction unit 103, and the feature amount classification unit 104 are omitted from the blood pressure measurement apparatus 100, thus reducing the size and mass of the blood pressure measurement apparatus 100 and making it possible to easily arrange the sensor at position that enables accurate measurement. This consequently reduces the burden on the user, and makes it possible to easily perform accurate blood pressure measurement.

The device of the present invention can also be realized by a computer and a program, and the program can be provided by being recorded on a recording medium or can be provided via a network.

Also, the above-described devices and the constituent portions thereof can be implemented by hardware configurations or combinations of hardware resources and software. In the case of combination configurations, the software is a program that is installed in a computer in advance via a network or with use of a computer-readable recording medium, and is executed by a processor of the computer in order to cause the computer to realize the functions of the above-described devices.

Note that this invention is not limited directly to the embodiments described above, and constituent elements can be changed without departing from the gist of the invention in the implementation phase. Also, various inventions can be formed through appropriate combinations of constituent elements disclosed in the above embodiments. For example, several of the constituent elements disclosed in the embodiments may be omitted. Furthermore, constituent elements from different embodiments may be combined with each other.

Also, portions or all of the above embodiments can also be described as noted below, but there is no limitation to the following content.

### Supplementary Note 1

A blood pressure measurement apparatus including a hardware processor and a memory,

the hardware processor being configured to
obtain time-series data indicating a blood pressure value that changes in conjunction with a heartbeat,
detect one or more surge sections that each include one surge from the time-series data,
extract one or more feature amounts for each of the surges, and
classify each of the surges based on the one or more feature amounts, and

the memory including
a storage unit that stores the classified surges.

### Supplementary Note 2

A blood pressure measurement method comprising the steps of:

with use of at least one hardware processor, obtaining time-series data indicating a blood pressure value that changes in conjunction with a heartbeat;

with use of at least one hardware processor, detecting one or more surge sections that each include one surge from the time-series data;

with use of at least one hardware processor, extracting one or more feature amounts for each of the surges; and

with use of at least one hardware processor, classifying each of the surges based on the one or more feature amounts.

1. A blood pressure measurement apparatus comprising:
a blood pressure measurement unit configured to obtain time-series data indicating, for each heartbeat, a blood pressure value that changes in conjunction with a heartbeat;
a detection unit configured to detect one or more surge sections that each include one surge from the time-series data;
an extraction unit configured to extract one or more feature amounts for each of the surges; and
a classification unit configured to classify each of the surges based on the one or more feature amounts,
wherein the extraction unit extracts a time period in which a surge is occurring, and an amount of change in blood pressure in the time period, as the one or more feature amounts.

2. The blood pressure measurement apparatus according to claim 1,
wherein the classification unit maps each of the surges in a space, the number of dimensions of the space being greater than or equal to 1 and less than or equal to the number of feature amounts, and each axis of the space corresponding to one of the feature amounts.

3. The blood pressure measurement apparatus according to claim 1,
wherein the extraction unit extracts, as the one or more feature amounts for each of the surges, a rise time from when the surge starts until when the surges reaches a peak, a fall time from when the surge reaches the peak until when the surge ends, and an amount of change of the blood pressure in the rise time.

4. The blood pressure measurement apparatus according to claim 1,
wherein letting a peak point be a local maximum value of at least one of a systolic blood pressure, a diastolic blood pressure, an average blood pressure, and a pulse pressure in the time-series data, letting a start point be a closest local minimum value before the peak point, and letting an end point be a point that is before a closest local minimum value after the peak point and that is when a difference from the closest local minimum value after the peak point has fallen to a certain value or lower, in a case where a difference between a blood pressure value at the peak point and a blood pressure value at the start point is greater than a threshold value, furthermore a time difference between the peak point and a start point prior thereto is greater than a first duration, and still furthermore a time difference between the peak point and the end point is greater than or equal to a second duration, the detection unit detects that a section from the start point to the end point with the peak point therebetween is a surge section.

5. The blood pressure measurement apparatus according to claim 1, further comprising:
a visualization unit configured to visualize each of the surges classified by the classification unit with use of the one or more feature amounts as one or more axes.

6. The blood pressure measurement apparatus according to claim 1, further comprising:
a calculation unit configured to calculate a statistic regarding the one or more feature amounts.

7. The blood pressure measurement apparatus according to claim 6, further comprising:
a visualization unit configured to visualize each of the surges classified by the classification unit with use of the statistic regarding the one or more feature amounts as one or more axes.

8. The blood pressure measurement apparatus according to claim 6, further comprising:
a judgment unit configured to judge a level of danger of a biological body based on a threshold value for comparison with the statistic, the threshold value being associated with a level of danger that is set in advance in correspondence with the statistic.

9. The blood pressure measurement apparatus according to claim 8, further comprising:
a visualization unit configured to visualize each of the surges in accordance with the level of danger, wherein a danger reference corresponding to the threshold value is set in order to judge whether or not a surge is dangerous.

10. The blood pressure measurement apparatus according to claim 1, further comprising:
a judgment unit configured to judge whether a detected surge corresponds to a time in a dosage period based on a dosage record.

11. The blood pressure measurement apparatus according to claim 10, further comprising:
a visualization unit configured to visualize each of the surges in accordance with whether or not the surge corresponds to a time in the dosage period.

12. The blood pressure measurement apparatus according to claim 5,
wherein the blood pressure measurement unit obtains the time-series data for each of a plurality of users, and
the visualization unit visualizes each of the surge for each of the users.

13. The blood pressure measurement apparatus according to claim 1, further comprising:
a frequency calculation unit configured to calculate an occurrence frequency of the surges; and
an index calculation unit configured to calculate an apnea-hypopnea index based on the occurrence frequency with reference to a relationship between the surge occurrence frequency and an apnea-hypopnea index, the relationship being acquired in advance.

14. A blood pressure measurement method comprising the steps of:
obtaining time-series data indicating, for each heartbeat, a blood pressure value that changes in conjunction with a heartbeat;
detecting one or more surge sections that each include one surge from the time-series data;
extracting one or more feature amounts for each of the surges; and
classifying each of the surges based on the one or more feature amounts,
wherein when extracting the one or more feature amounts, a time period in which a surge is occurring, and an amount of change in blood pressure in the time period are extracted as the one or more feature amounts.

15. A program for causing a computer to function as the blood pressure measurement apparatus according to claim 1.
